SEQUENCE LISTING (4 sequences)

SEQ ID NO: 1 - RNA, 36 nt, synthetic construct
  source 1..36: mol_type = other RNA
tcaaaatggaaggttatacagcagccgaaaggctgc

SEQ ID NO: 2 - RNA, 22 nt, synthetic construct
  source 1..22: mol_type = other RNA
tgtataaccttccattttgagg

SEQ ID NO: 3 - RNA, 36 nt, synthetic construct; identical to SEQ ID NO: 1
  source 1..36: mol_type = other RNA
  modified_base 1: mod_base = OTHER; note = 2'-O-methyluridine with a 3'-phosphorothioate linkage
  modified_base 2: mod_base = cm
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = gm
  modified_base 13: mod_base = gm
  modified_base 14: mod_base = um
  modified_base 15: mod_base = um
  modified_base 16: mod_base = OTHER; note = 2'-O-methyladenosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25: mod_base = cm
  modified_base 26: mod_base = cm
  modified_base 27: mod_base = gm
  modified_base 28: mod_base = OTHER; note = 2'-aminodiethoxymethanol-adenine-N-acetylgalactosam ine
  modified_base 29: mod_base = OTHER; note = 2'-aminodiethoxymethanol-adenine-N-acetylgalactosam ine
  modified_base 30: mod_base = OTHER; note = 2'-aminodiethoxymethanol-adenine-N-acetylgalactosam ine
  modified_base 31: mod_base = gm
  modified_base 32: mod_base = gm
  modified_base 33: mod_base = cm
  modified_base 34: mod_base = um
  modified_base 35: mod_base = gm
  modified_base 36: mod_base = cm

SEQ ID NO: 4 - RNA, 22 nt, synthetic construct
  source 1..22: mol_type = other RNA
  modified_base 1: mod_base = OTHER; note = 4'-O-monomethylphosphonate-2'-O-methyluridine with a 3'-phosphorothioate linkage
  modified_base 2: mod_base = OTHER; note = 2'-fluoroguanosine with a 3'-phosphorothioate linkage
  modified_base 3: mod_base = OTHER; note = 2'-fluorouridine with a 3'-phosphorothioate linkage
  modified_base 4: mod_base = OTHER; note = 2'-fluoroadenosine
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = cm
  modified_base 10: mod_base = OTHER; note = 2'-fluorouridine
  modified_base 11: mod_base = um
  modified_base 12: mod_base = cm
  modified_base 13: mod_base = cm
  modified_base 14: mod_base = OTHER; note = 2'-fluoroadenosine
  modified_base 15: mod_base = um
  modified_base 16: mod_base = um
  modified_base 17: mod_base = um
  modified_base 18: mod_base = um
  modified_base 19: mod_base = gm
  modified_base 20: mod_base = OTHER; note = 2'-O-methyladenosine with a 3'-phosphorothioate linkage
  modified_base 21: mod_base = OTHER; note = 2'-O-methylguanosine with a 3'-phosphorothioate linkage
  modified_base 22: mod_base = gm
tgtataaccttccattttgagg

(19) United States
(12) Patent Application Publication
(10) Pub. No.: US 2026/0139242 A1
(43) Pub. Date: May 21, 2026

(54) APPARATUSES FOR AND METHODS OF CONCENTRATING BIOMOLECULES

(71) Applicant: ELI LILLY AND COMPANY, Indianapolis, IN (US)

(72) Inventors: McKensie Lee MASON, Greenwood, IN (US); John Fletcher MOOMAW, Indianapolis, IN (US); Ken Kangyi QIAN, Carmel, IN (US); Lina WU, Cambridge, MA (US)

(21) Appl. No.: 19/120,280

(22) PCT Filed: Oct. 13, 2023
(86) PCT No.: PCT/US2023/076786

§ 371 Date: Apr. 11, 2025

Related U.S. Application Data
(60) Provisional application No. 63/379,530, filed on Oct. 14, 2022.

(51) Int. Cl. C12N 15/10

(52) U.S. Cl. CPC C12N 15/1017

(57) ABSTRACT

An apparatus is described for concentrating biomolecules in solution, such as oligonucleotide-containing solutions, via tangential flow filtration (TFF), where the apparatus can concentrate the oligonucleotides to a concentration>100 mg/mL. Also described are methods of concentrating a biomolecule-containing solution, such as oligonucleotide-containing solutions, via TFF for high dose/low volume applications.

### REFERENCE TO SEQUENCE LISTING SUBMITTED ELECTRONICALLY

The disclosure is filed along with a Sequence Listing in ST.26 XML format. The Sequence Listing is provided as a file titled “30194_US_PRI” created 5 Oct. 2022 and is 28 kilobytes (kb) in size. The Sequence Listing information in the ST.26 XML format is incorporated herein by reference in its entirety.

### TECHNICAL FIELD

The disclosure relates to chemistry and engineering, and more particularly it relates to apparatuses for and methods of concentrating biomolecules in solutions, such as single-stranded (ss) or double-stranded (ds) oligonucleotides, for use as a drug substance (DS) in the manufacture of a drug product (DP).

### BACKGROUND

Therapeutic oligonucleotides are a newer modality for treating and preventing diseases and disorders. Therapeutic oligonucleotide synthesis may involve a number of upstream and downstream steps such as, for example, synthesizing, cleaving and deprotecting, purifying, and concentrating. Of particular interest herein is the concentrating of therapeutic oligonucleotides in solution, which are required as a DS in the manufacture of a DP or other pharmaceutical composition.

A number of methods of concentrating oligonucleotides in solution are known such as chromatography, dialysis, evaporation, precipitation and ultrafiltration/diafiltration (UF/DF). At present, these methods of concentrating oligonucleotides do not reliably achieve final oligonucleotide concentrations in solution greater than about 100 mg/mL. With regard to UF/DF, especially a form known as tangential flow filtration (TFF), several factors limit its ability to achieve high final concentrations such as, for example, increasing viscosity/osmotic pressure of a retentate solution with increasing oligonucleotide concentration and fouling of the membrane with increasing oligonucleotide concentration. Highly concentrated oligonucleotides, however, are required for formulating and dosing individuals in need thereof with a minimal volume of solution.

Therefore, there is a need for apparatuses for and methods of concentrating biomolecule-containing solutions, such as oligonucleotide-containing solutions, to a concentration>100 mg/mL.

### BRIEF SUMMARY

To address this need, the disclosure first describes an apparatus for concentrating biomolecules in a solution such as oligonucleotides for high dose/low volume administration. The apparatus includes:
i. a first reservoir in fluid communication with a first side of a membrane, where the first reservoir is a retentate feed, and where the membrane has a molecular weight cutoff (MWCO) of <about 5 kDa;
ii. a second reservoir in fluid communication with a second opposing side of the membrane, where the second reservoir is a draw solution feed and a permeate collector;
iii. a first feed pump for cycling a retentate past the first side of the membrane;
iv. a second feed pump for cycling the draw solution or draw solution/permeate mixture past the second opposing side of the membrane;
v. a first balance with stir plate, where the first balance with stir plate is for stirring the retentate in the first reservoir and measuring the retentate;
vi. a second balance with stir plate, where the second balance with stir plate is for stirring the draw solution/permeate mixture in the second reservoir and measuring the draw solution/permeate mixture; and
vii. a transmembrane pressure (TMP) controller.

In some instances, the MWCO is at least about 1 kDa. In other instances, the MWCO is at least about 2 kDa.

In some instances, the membrane further includes a surface area of at least about 0.02 m² to about 0.1 m².

In some instances, the membrane is a polyethersulfone (PES) membrane or a regenerated cellulose membrane.

In addition, the disclosure describes a method of concentrating an oligonucleotide-containing solution that includes at least steps of:
a. cycling a first solution past a first side of a membrane, where the first solution is a retentate having a starting oligonucleotide concentration of <about 95 mg/mL and a pH between about 6 to about 7, and where the membrane has a MWCO of <about 5 kDa;
b. cycling a second solution past a second opposing side of the membrane, where the second solution is a permeate having a pH between about 6 to about 8 and an ionic strength greater that the first solution;
c. maintaining a TMP across the membrane between about 30 psi to about 45 psi; and
d. maintaining the cycling of the retentate and the permeate past the first and second opposing membrane sides, respectively, until the first solution has a final oligonucleotide concentration of at least about 100 mg/mL.

In some instances, the starting oligonucleotide concentration is at least about 20 mg/mL.

In some instances, the first solution is water.

In some instances, the MWCO is at least about 1 kDa. In other instances, the MWCO is at least about 2 kDa.

In some instances, the membrane further includes a surface area of at least about 0.02 m² to about 0.1 m².

In some instances, the membrane is a PES membrane or a regenerated cellulose membrane.

In some instances, the ionic strength of the second solution is between about 0.01 mol/L to about 2 mol/L.

In some instances, the second solution includes a NaCl concentration between about 0.01 M to about 2 M.

In some instances, the second solution comprises a MgCl₂ concentration between 0.03 M about to about 2.0 M.

In some instances, the second solution comprises a dextran sulfate concentration between 100 mg/mL about to about 200 mg/mL.

In some instances, the ionic strength of the second solution is increased during the cycling. In other instances, the ionic strength of the second solution is maintained during the cycling.

In some instances, the final oligonucleotide concentration is from about 150 mg/mL to about 200 mg/mL. In other instances, the final oligonucleotide concentration is from about 200 mg/mL to about 250 mg/mL. In yet other instances, the final oligonucleotide concentration is from about 250 mg/mL to about 300 mg/mL.

In some instances, the final oligonucleotide concentration is between about 150 mg/mL to about 300 mg/mL.

Moreover, the disclosure describes a composition including oligonucleotides, such as therapeutic oligonucleotides, at a concentration>100 mg/mL. In some instances, the composition is solution. In other instances, the composition is a lyophilized powder.

An advantage of the apparatuses herein is that they do not require a complicated setup as compared to a conventional TFF setup and thus are technologically and economically feasible.

An advantage of the methods herein is that they allow for a significant increase in oligonucleotide concentration in a solution as compared to the concentration that can be achieved in a conventional TFF setup.

### BRIEF DESCRIPTION OF THE DRAWINGS

The advantages, effects, features, and objects other than those set forth above will become more readily apparent when consideration is given to the detailed description below. Such detailed description refers to the following drawing(s), where:

FIGS. 1a to 1d show apparatuses for concentrating ds therapeutic oligonucleotides, where blue dashed lines represent oligonucleotide (e.g., siRNA) solution/retentate flow path (FIGS. 1a and 1c) and where green dashed lines represent draw solution/permeate flow path (FIGS. 1b and 1d). FIGS. 1a and 1c depict a typical TFF flow path of a retentate being concentrated as it is recirculated through a retentate side of a membrane; and FIGS. 1b and 1d depict an exemplary TFF flow path having an added pathway for a permeate to recirculate through a permeate side of a membrane.

FIG. 2 shows retentate scale weight and permeate flux over time following typical TFF. In FIG. 2, retentate scale weight and permeate flux decreased over time during the course of the concentration experiment. TMP was increased from 40 psi to 42 psi after 30 min. The concentration of the final retentate was 101 mg/mL.

FIG. 3 shows retentate scale weight over time following TFF using draw solutions having varied NaCl concentrations. In FIG. 3, the runs ended when the retentate scale weight remained constant for 30 min or when the minimum reservoir volume was reached (for the 500 mM draw solution sample). Data was adjusted to start at the same weight at a time of 0 to account for slight variations in the delay time between the start of data collection and the start of the run for each experiment.

FIG. 4 shows permeate flux data collected during TFF over time for the first hour (exponential decay curves were fitted to the data starting at the initial highest permeate flux value).

FIG. 5 shows retentate scale weight collected over time during TFF. In FIG. 5, the runs were ended when the retentate scale weight remained constant for 30 min or when the minimum reservoir volume was reached (for the 167 mM MgCl₂ draw solution sample). Data was adjusted to start at the same weight at a time of 0 to account for slight variations in the delay time between the start of data collection and the start of the run for each experiment.

FIG. 6 shows retentate scale weight collected over time during TFF. In FIG. 6, the runs were ended when the retentate scale weight remained constant for 30 min. Data was adjusted to start at the same weight at a time of 0 to account for slight variations in the delay time between the start of data collection and the start of the run for each experiment.

### DETAILED DESCRIPTION

### Overview

Therapeutic oligonucleotides, especially activating RNA (aRNA)-, editing RNA (eRNA)- and inhibiting RNA (iRNA)-based therapeutic oligonucleotides, are an emerging class of biomolecules. Current apparatuses and methods for concentrating therapeutic oligonucleotides via TFF, especially ds oligonucleotides, are hampered by inherent characteristics of the oligonucleotides such as flow-induced elongation, which requires using membranes with small MWCOs and which also reduces flux. Additionally, oligonucleotides are highly negatively charged, which rapidly increases osmotic pressure while concentrating. Moreover, the increased osmotic pressure counteracts TMP, further slowing flux.

As shown herein, these disadvantages can be addressed by incorporating a forward osmosis process with a permeate (i.e., draw solution) having a high ionic strength. The apparatuses and methods herein provide high concentration therapeutic oligonucleotides (i.e., ≥100 mg/mL) in a low volume solution (i.e., ≤2 mL).

### Abbreviations and Definitions

Unless defined otherwise, all technical and scientific terms used herein have the same meaning as commonly understood by one of skill in the art to which the disclosure pertains. Although any methods and materials similar to or equivalent to those described herein can be used in the practice or testing of the methods herein, the preferred methods and materials are described herein.

Additionally, reference to an element by the indefinite article “a” or “an” does not exclude the possibility that more than one element is present, unless the context clearly requires that there be one and only one element. The indefinite article “a” or “an” thus usually means “at least one.”

Moreover, use of “including,” as well as other forms, such as “including but not limited, “include,” “includes” and “included,” is not limiting.

Certain abbreviations used herein are as follows:

“ADAR” refers to adenosine deaminase acting on RNA enzyme; “API” refers to active pharmaceutical ingredient; “aRNA” refers to activating RNA; “ASO” refers to antisense oligonucleotide; “DNA” refers to deoxyribonucleic acid; “DP” refers to drug product; “ds” refers to double-stranded”; “DS” refers to drug substance; “DsiRNA” refers to Dicer substrate interfering RNA; “eRNA” refers to editing RNA; “H₂O” refers to water; “hr” refers to hour(s); “iRNA” refers to inhibiting RNA; “kDa” refers to kilodalton(s); “L” refers to liter(s); “mg” refers to milligram; “MgCl₂” refers to magnesium chloride; “MgSO₄” refers to magnesium sulfate; “min” refers to minute(s); “mol” refers to moles; “mRNA” refers to messenger RNA; “miRNA” refers to microRNA; “mL” refers to milliliter(s); “MW” refers to molecular weight; “MWCO” refers to molecular-weight cut-off; “Na₃C₆H₅O₇” refers to sodium citrate; “NaCl” refers to sodium chloride; “Na₂SO₄” refers to sodium sulfate; “PES” refers to polyethersulfone; “psi” refers to pounds per square inch; “RISC” refers to RNA-induced silencing complex; “RITA” refers to RNA-induced transcriptional activation; “RNA” refers to ribonucleic acid; “rRNA” refers to ribosomal RNA; “shRNA” refers to short hairpin RNA; “siRNA” refers to small interfering RNA; “SPS” refers to solid-phase synthesis; “ss” refers to single-stranded; “TFF” refers to tangential flow filtration; “TMP” refers to transmembrane pressure; “tRNA” refers to transfer RNA; and “UV” refers to ultraviolet.

Certain definitions used herein are defined as follows:

As used herein, “about” means within a statistically meaningful range of a value or values such as, for example, a stated area, concentration, length, molecular weight, pH, sequence similarity, time frame, temperature, volume, etc. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, “activating RNA” or “aRNA” means a nucleic acid that contains RNA and that mediates the targeted activation of a promoter or other non-coding transcript of a RNA transcript via a RNA-induced transcriptional activation (RITA) complex pathway. aRNA's typically are ds. The aRNA activates, increases, modulates or upregulates expression of a target nucleotide sequence in a cell.

As used herein, “biomolecule” and the like means a molecule or compound that includes or incorporates amino acids, carbohydrates, lipids and/or nucleotides. Examples of biomolecules of interest herein include, but are not limited to, nucleic acids (e.g., oligonucleotides and polynucleotides), peptides, polypeptides and proteins.

As used herein, “deoxyribonucleotide” means a nucleotide having a hydrogen in place of a hydroxyl at the 2′ position of its pentose sugar when compared with a ribonucleotide. A modified deoxyribonucleotide has one or more modifications or substitutions of atoms other than hydroxyl at the 2′ position, including modifications or substitutions in or of the nucleobase, sugar, or phosphate group.

As used herein, “draw solution” means a solution circulated on a permeate side of a membrane with the purpose of creating an osmotic pressure/ionic strength difference across the membrane to facilitate or “draw” further permeation of water.

As used herein, “drug product” or “DP” means a finished product of any therapeutic agent, such as a therapeutic oligonucleotide, that is available in the market and is ready to use generally, but not necessarily, in association with one or more other pharmaceutically acceptable ingredients.

As used herein, “drug substance” or “DS” means an active ingredient, such as a therapeutic oligonucleotide, that is intended to furnish pharmacological activity or other direct effect in diagnosing, curing, mitigating, treating and/or preventing disease or affecting the structure or any function of the body, but does not include intermediates used in the synthesis of such ingredient. DS also is known as an active pharmaceutical ingredient (API). A DS is used in preparing a DP.

As used herein, “editing RNA” or “eRNA” means a nucleic acid that contains RNA and that mediates inserting, deleting and even base substituting of nucleotides within the target nucleotide sequence. RNAe has been observed in a number of different types of RNA such as, for example, messenger RNA (mRNA), microRNA (miRNA), transfer RNA (tRNA) and ribosomal RNA (rRNA). RNAe is enzymatically mediated either by exogenously supplying adenosine deaminase acting on RNA enzyme (ADAR) or by directing an endogenous ADAR to a specific site in a target RNA nucleotide sequence, typically involve editing at a single nucleotide site by directing the ADAR to that site through complimentary oligonucleotides. eRNA's typically are ss.

As used herein, “inhibiting RNA” or “iRNA” means a nucleic acid that contains RNA and that mediates the targeted cleavage of a RNA transcript via RNA interference, for example, through a RNA-induced silencing complex (RISC) pathway. Some iRNA's are single-stranded (ss) and other iRNA's are ds and have a sense strand and an antisense strand, where the sense strand and the antisense strand form a duplex. The iRNA directs sequence-specific degradation of mRNA via RNA interference. The iRNA attenuates, inhibits, modulates or reduces expression of a target nucleotide sequence in a cell. Examples of iRNA include, but are not limited to, an antisense oligonucleotide (ASO), Dicer substrate interfering RNA (DsiRNA), miRNA, short hairpin RNA (shRNA) or small interfering RNA (siRNA).

As used herein, “nucleotide” means an organic compound having a nucleoside (a nucleobase such as, for example, adenine, cytosine, guanine, thymine, or uracil; and a pentose sugar such as, for example, ribose or 2′-deoxyribose) and a phosphate group. A nucleotide can serve as a monomeric unit of nucleic acid polymers such as deoxyribonucleic acid (DNA) and ribonucleic acid (RNA).

As used herein, “oligonucleotide” means a short nucleic acid compound (e.g., a polymer of less than about 100 nucleotides in length) and may include deoxyribonucleotides (or modified deoxyribonucleotides), ribonucleotides (or modified ribonucleotides) or both. Likewise, an oligonucleotide may be ss or ds and thus may or may not have duplex regions.

As used herein, “synthetic” refers to a nucleic acid or other compound that is artificially synthesized (e.g., using a machine such as, for example, a solid phase nucleic acid synthesizer) or that is otherwise not derived from a natural source (e.g., a cell or organism) that normally produces the nucleic acid or other compound.

As used herein, “ribonucleotide” means a nucleotide having a ribose as its pentose sugar, which contains a hydroxyl group at its 2′ position. A modified ribonucleotide is a ribonucleotide having one or more modifications or substitutions of atoms other than hydrogen at the 2′ position, including modifications or substitutions in or of the nucleobase, sugar, or phosphate group.

As used herein, “therapeutic oligonucleotide” means a ss or ds nucleic acid that has a therapeutic application (i.e., application in treating a disease). Such a nucleic acid typically contains one or more modified nucleotide residues or linkages and also can include a targeting ligand and/or delivery vehicle. Examples of therapeutic oligonucleotides include, but are not limited to, aRNA, eRNA and iRNA. Specific examples of therapeutic oligonucleotides include, but are not limited to, ASOs, aptamers, short activating RNAs (saRNAs), siRNAs, miRNAs and decoys.

### Apparatuses

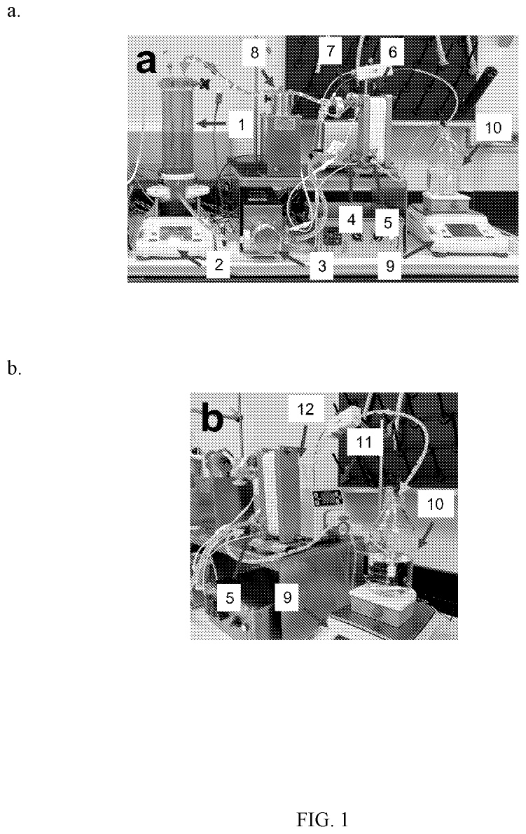

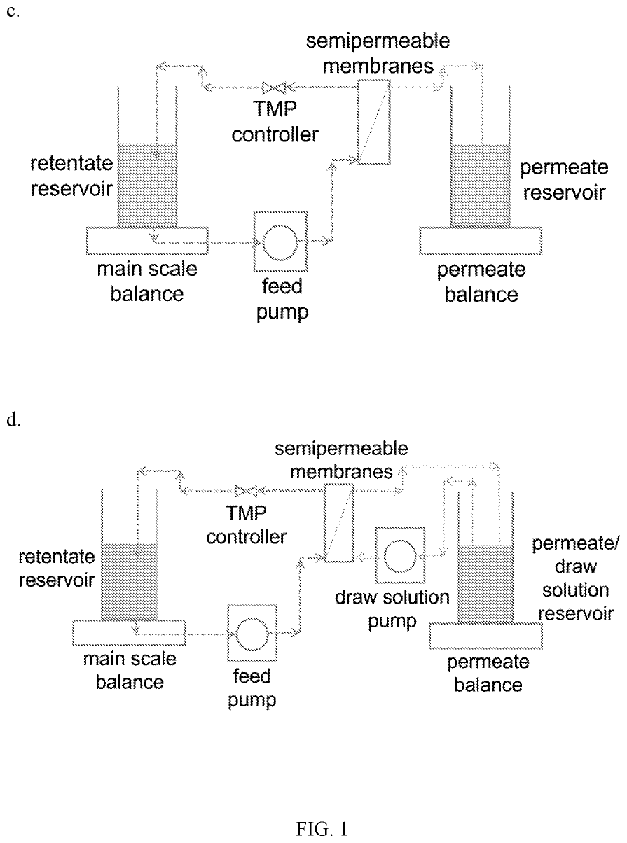

A typical TFF setup is shown in FIG. 1a. A solution to be concentrated, such as an oligonucleotide-containing solution, such as a ds oligonucleotide-containing solution, is loaded in reservoir (1) and pumped via a feed pump (3) through the flow path shown in blue. The solution enters a membrane cassette (6) through a retentate inlet port (4), flows across or tangential to a semipermeable membrane of the membrane cassette (6), then exits through a permeate outlet port (7) to continue back to the reservoir (1). The semipermeable membrane of the membrane cassette (6) has a MWCO smaller than the molecule to be concentrated (e.g., an oligonucleotide such as a ds therapeutic oligonucleotide), so that only the solvent passes through and is collected in a separate permeate container (10). The molecule to be concentrated is retained in a retentate and is concentrated in a retentate reservoir (1) as solvent crosses the membrane. As the retentate is being concentrated, a weight recorded on a retentate scale (2) decreases while a weight of a permeate scale (9) increases. Permeation is facilitated and driven by a TMP controller across the semipermeable membrane of the membrane cassette (6). TMP is defined as the average applied pressure from the retentate to the permeate sides of the membrane and is varied with a TMP controller (8), which presses on tubing to apply pressure.

An exemplary TFF setup of this disclosure including a flow path for a draw solution is shown in FIGS. 1b and 1d, which build upon the setup in FIGS. 1a and 1c. Here, a ss or ds oligonucleotide-containing solution is loaded into the reservoir (1) and is cycled at a constant feed flow rate along a retentate flow path (blue). A controlled TMP is applied across the membrane to drive H₂O out through a permeate outlet valve (12) and into a separate, permeate container (10). As H₂O permeates the membrane, the weight of a retentate scale (2) decreases while the weight of a permeate scale (9) increases. In contrast to the TFF setup shown in FIG. 1a, a high ionic strength draw solution is added to the permeate container (10) to facilitate continued H₂O permeation and is cycled into an inlet (5) at a constant flow rate by a draw solution pump (11) through the permeate side of the membrane, exiting the membrane holder through the permeate outlet valve (12) and returning to the permeate collection vessel, thereby driving the ds oligonucleotide concentration to a greater level than without the high ionic strength draw solution.

### Methods

The methods can include the steps described herein, and these maybe be, but not necessarily, carried out in the sequence as described. Other sequences, however, also are conceivable. Moreover, individual or multiple steps may be carried out either in parallel and/or overlapping in time and/or individually or in multiply repeated steps. Furthermore, the methods may include additional, unspecified steps.

Moreover, the oligonucleotides can be prepared by any method known in the art such as, for example, solid-phase synthesis (SPS) individual strands, which then optionally can undergo additional steps for purification, solvent exchange, de-salting and concentration prior to and/or following annealing into a duplex in H₂O for ds oligonucleotides.

Briefly, a method of concentrating an oligonucleotide-containing solution can include a step of cycling a first solution past a first side of a membrane, where the first solution is a retentate having a starting concentration of oligonucleotide of ≤95 mg/mL and a pH of about 6 to about 7, and where the membrane has a surface area from about 0.02 m² to about 0.1 m² and/or a MWCO of <5 kDa.

In some instances, the oligonucleotide is a ss oligonucleotide. In other instances, the oligonucleotide is a ds oligonucleotide.

In some instances, the starting oligonucleotide concentration of the first solution is <about 95 mg/mL. In other instances, the starting oligonucleotide concentration is between about 5 mg/mL to about 95 mg/mL, about 10 mg/mL to about 90 mg/mL, about 15 mg/mL to about 85 mg/mL, about 20 mg/mL to about 80 mg/mL, about 25 mg/mL to about 75 mg/mL, about 30 mg/mL to about 70 mg/mL, about 35 mg/mL to about 65 mg/mL, about 40 mg/mL to about 60 mg/mL, about 45 mg/mL to about 55 mg/mL, or about 50 mg/mL. In yet other instances, the starting oligonucleotide concentration is from about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75, mg/mL, about 80 mg/mL, about 85 mg/mL, about 90 mg/mL, or even about 95 mg/mL. In yet other instances, the starting oligonucleotide concentration is >about 95 mg/mL.

In some instances, the pH of the first solution is about 6.0, about 6.1, about 6.2, about 6.3, about 6.4, about 6.5, about 6.6, about 6.7, about 6.8, about 6.9 or about 7.0.

In some instances, the first solution is H₂O.

In some instances, the MWCO of the membrane is >about 1 kDa. In other instances, the MWCO is between about 1 kDa to about 5 kDa, about 2 kDa to about 4 kDa, or about 3 kDa. In yet other instances, the MWCO is about 1 kDa, about 1.5 kDa, about 2 kDa, about 2.5 kDa, about 3 kDa, about 3.5 kDa, about 4 kDa, about 4.5 kDa or about 5 kDa. In certain instances, the MWCO is about 2 kDa. In certain other instances, the MWCO is <3 kDa.

In some instances, the membrane is a PES membrane or a regenerated cellulose membrane.

In addition, the methods can include a step of cycling a second solution past a second opposing side of the membrane, where the second solution is a draw solution having an ionic strength greater than the first solution and a pH of about 6 to about 8. It should be noted that as the draw solution cycles through, it becomes a draw solution/permeate mixture as H₂O is drawn from the first solution.

In some instances, the pH of the second solution is about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9 or about 8.0. In other instances, the pH of the second solution is the same as the pH of the first solution. In yet other instances, the pH of the second solution is different from the pH of the first solution.

In some instances, the ionic strength of the second solution is >about 0.01 mol/L. In other instances, the ionic strength is between about 0.01 mol/L to about 2 mol/L. In yet other instances, the ionic strength of the second solution is between about 0.05 mol/L to about 1.95 mol/L, about 0.1 mol/L to about 1.9 mol/L, about 0.15 mol/L to about 1.85 mol/L, about 0.2 mol/L to about 1.80 mol/L, about 0.25 mol/L to about 1.75 mol/L, about 0.3 mol/L to about 1.7 mol/L, about 0.35 mol/L to about 1.65 mol/L, about 0.4 mol/L to about 1.6 mol/L, about 0.45 mol/L to about 1.55 mol/L, about 0.5 mol/L to about 1.5 mol/L, about 0.55 mol/L to about 1.45 mol/L, about 0.6 mol/L to about 1.4 mol/L, about 0.65 mol/L to about 1.35 mol/L, about 0.7 mol/L to about 1.3 mol/L, about 0.75 mol/L to about 1.25 mol/L, about 0.8 mol/L to about 1.2 mol/L, about 0.85 mol/L to about 1.15 mol/L, about 0.9 mol/L to about 1.1 mol/L, about 0.95 mol/L to about 1.05 mol/L, or about 1.0 mol/L. Alternatively, the ionic strength is about 0.01 mol/L, about 0.05 mol/L, about 0.1 mol/L, about 0.15 mol/L, about 0.2 mol/L, about 0.25, about 0.3 mol/L, about 0.35 mol/L, about 0.4 mol/L, about 0.45 mol/L, about 0.5 mol/L, about 0.55 mol/L, about 0.6 mol/L, about 0.65 mol/L, about 0.7 mol/L, about 0.75 mol/L, about 0.8 mol/L, about 0.85 mol/L, about 0.9 mol/L, about 0.95 mol/L, about 1.0 mol/L, about 1.1 mol/L, about 1.15 mol/L, about 1.2 mol/L, about 1.25 mol/L, about 1.3 mol/L, about 1.35 mol/L, about 1.4 mol/L, about 1.45 mol/L, about 1.5 mol/L, about 1.55 mol/L, about 1.6 mol/L, about 1.65 mol/L, about 1.7 mol/L, about 1.75 mol/L, about 1.8 mol/L, about 1.85 mol/L, about 1.9 mol/L, about 1.95 mol/L or about 2.0 mol/L.

In some instances, the second solution includes NaCl at a concentration between about 0.01 M to about 2 M NaCl. In other instances, the NaCl concentration is between about 0.05 M to about 1.95 M, about 0.1 M to about 1.90 M, about 0.15 M to about 1.85 M, about 0.2 M to about 1.8 M, about 0.25 M to about 1.75 M, about 0.3 M to about 1.7 M, about 0.35 M to about 1.65 M, about 0.4 M to about 1.6 M, about 0.45 M to about 1.55 M, about 0.5 M to about 1.5 M, about 0.55 M to about 1.45 M, about 0.6 M to about 1.4 M, about 0.65 M to about 1.35 M, about 0.7 M to about 1.3 M, about 0.75 M to about 1.25 M, about 0.8 M to about 1.2 M, about 0.85 M to about 1.15 M, about 0.9 M to about 1.1 M, about 0.95 M to about 1.05 M, or about 1.0 M. Alternatively, the NaCl concentration is about 0.01 M, about 0.15 M, about 0.2 M, about 0.25 M, about 0.3 M, about 0.35 M, about 0.4 M, about 0.45 M, about 0.5 M, about 0.55 M, about 0.6 M, about 0.65 M, about 0.7 M, about 0.75 M, about 0.8 M, about 0.85 M, about 0.9 M, about 0.95 M, about 1.0 M, about 1.05 M, about 1.1 M, about 1.15 M, about 1.2 M, about 1.25 M, about 1.3 M, about 1.35 M, about 1.4 M, about 1.45 M, about 1.5 M, about 1.55 M, about 1.6 M, about 1.65 M, about 1.7 M, about 1.75 M, about 1.8 M, about 1.85 M, about 1.9 M, about 1.95 M or about 2.0 M.

In addition, the methods can include a step of maintaining a TMP across the membrane of between about 30 psi to about 45 psi at least until there is no longer any change in the weight scales or until a minimum volume of the reservoir is reached. In other instances, TMP can be increased over time, where an upper limit is dictated by the instrument pressure limits. In some instances, the TMP is between about 31 psi to about 44 psi, about 32 psi to about 43 psi, about 33 psi to about 42 psi, about 34 psi to about 41 psi, about 35 psi to about 40 psi, about 36 psi to about 39 psi or about 37 psi to about 38 psi. In other instances, the TMP is between about 30 psi to about 32 psi, about 32 psi to about 34 psi, about 34 psi to about 36 psi, about 36 psi to about 38 psi, about 38 psi to about 40 psi, about 40 psi to about 42 psi, about 42 psi to about 44 psi, about 30 psi to about 35 psi, about 35 psi to about 40 psi or about 40 psi to about 45 psi. In yet other instances, the TMP is about 30 psi, about 31 psi, about 32 psi, about 33 psi, about 34 psi, about 35 psi, about 36 psi, about 37 psi, about 38 psi, about 39 psi, about 40 psi, about 41 psi, about 42 psi, about 43 psi, about 44 psi or about 45 psi.

In some instances, the first solution is flowed past the first side of the membrane at a rate from about 0.1 L/min to about 1.5 L/min, about 0.2 L/min to about 1.4 L/min, about 0.3 L/min to about 1.3 L/min, about 0.4 L/min to about 1.2 L/min, about 0.5 L/min to about 1.1 L/min, about 0.6 L/min to about 1.0 L/min, about 0.7 L/min to about 0.9 L/min, or about 0.8 L/min. In other instances, the rate is from about 0.1 L/min to about 0.25 L/min, about 0.25 L/min to about 0.5 L/min, about 0.5 L/min to about 0.75 L/min, about 0.75 L/min to about 1.0 L/min, about 1.0 L/min to about 1.25 L/min, or about 1.25 L/min to about 1.5 L/min. In yet other instances, the rate is about 0.1 L/min, about 0.2 L/min, about 0.3 L/min, about 0.4 L/min, about 0.5 L/min, about 0.6 L/min, about 0.7 L/min, about 0.8 L/min, about 0.9, about 1.0 L/min, about 1.1 L/min, about 1.2 L/min, about 1.3 L/min, about 1.4 L/min or about 1.5 L/min.

In some instances, the second solution is flowed past the second opposing side of the membrane at a rate from about 1.0 L/min to about 15.0 L/min, about 1.5 L/min to about 14.5 L/min, about 2.0 L/min to about 14.0 L/min, about 2.5 L/min to about 13.5 L/min, about 3.0 L/min to about 13.0 L/min, about 3.5 L/min to about 12.5 L/min, about 4.0 L/min to about 12.0 L/min, about 4.5 L/min to about 11.5 L/min, about 5.0 L/min to about 11.0 L/min, about 5.5 L/min to about 10.5 L/min, about 6.0 L/min to about 10.0 L/min, about 6.5 L/min to about 9.5 L/min, about 7.0 L/min to about 9.0 L/min, about 7.5 L/min to about 8.5 L/min, or about 8.0 L/min. In other instances, the rate is from about 1.0 L/min to about 1.5 L/min, about 1.5 L/min to about 2.0 L/min, about 2.0 L/min to about 2.5 L/min, about 2.5 L/min to about 3.0 L/min, about 3.0 L/min to about 3.5 L/min, about 3.5 L/min to about 4.0 L/min, about 4.0 L/min to about 4.5 L/min, about 4.5 L/min to about 5.0 L/min, about 5.0 L/min to about 5.5 L/min, about 5.5 L/min to about 6.0 L/min, about 6.0 L/min to about 6.5 L/min, about 6.5 L/min to about 7.0 L/min, about 7.0 L/min to about 7.5 L/min, about 7.5 L/min to about 8.0 L/min, about 8.0 L/min to about 8.5 L/min, about 8.5 L/min to about 9.0 L/min, about 9.0 L/min to about 9.5 L/min, about 9.5 L/min to about 10.0 L/min, about 10.0 L/min to about 10.5 L/min, about 10.5 L/min over 11.0 L/min, about 11.0 L/min to about 11.5 L/min, about 11.5 L/min to about 12.0 L/min, about 12.0 L/min to about 12.5 L/min, about 12.5 L/min to about 13.0 L/min, about 13.0 L/min to about 13.5 L/min, about 13.5 L/min to about 14.0 L/min, about 14.0 L/min to about 14.5 L/min, or about 14.5 L/min to about 15.0 L/min. In certain other instances, the rate is about 1.0 L/min, about 1.5 L/min, about 2.0 L/min, about 2.5 L/min, about 3/0 L/min, about 3.5 L/min, about 4.0 L/min, about 4.5 L/min, about 5.0 L/min, about 5.5 L/min, about 6.0 L/min, about 6.5 L/min, about 7.0 L/min, about 7.5 L/min, about 8.0 L/min, about 8.5 L/min, about 9.0 L/min, about 9.5 L/min, about 10.0 L/min, about 10.5 L/min, about 11.0 L/min, about 11.5 L/min, about 12.0 L/min, about 12.5 L/min, about 13.0 L/min, about 13.5 L/min, about 14.0 L/min, about 14.5 L/min or about 15.0 L/min.

In some instances, the ionic strength of the second solution is increased during the cycling. In other instances, the ionic strength of the second solution is increased by about 1% to about 10% as compared to its starting ionic strength. In certain instances, the ionic strength of the second solution is increased by about 1%, about 2%, about 3%, about 4%, about 5%, about 6%, about 7%, about 8%, about 9% or about 10% as compared to its starting ionic strength.

Furthermore, the methods can include a step of maintaining the cycling of the retentate and the permeate past the first and the second opposing membrane sides until the ds oligonucleotide in the first solution is at a final concentration of at least about 100 mg/mL to about 150 mg/mL. In some instances, the final concentration is between about 150 mg/mL to about 300 mg/mL, about 160 mg/mL to about 290 mg/mL, about 170 mg/mL to about 280 mg/mL, about 180 mg/mL to about 270 mg/mL, about 190 mg/mL to about 260 mg/mL, about 200 mg/mL to about 250 mg/mL, about 210 mg/mL toa bout 240 mg/mL, or about 220 mg/mL to about 230 mg/mL. In other instances, the final concentration is about 150 mg/mL, about 160 mg/mL, about 170 mg/mL, about 180 mg/mL, about 190 mg/mL, about 200 mg/mL, about 210 mg/mL, about 220 mg/mL, about 230 mg/ml, about 240 mg/mL, about 250 mg/mL, about 260 mg/mL, about 270 mg/mL, about 280 mg/mL, about 290 mg/mL or about 300 mg/mL. In yet other instances, the final concentration is >300 mg/mL.

### EXAMPLES

The following non-limiting examples are offered for purposes of illustration, not limitation.

### Example 1: TFF of a Ds Oligonucleotide-Containing Solution without a Draw Solution

Purpose: To concentrate a solution of ds oligonucleotides to a concentration greater than 100 mg/mL.

### Methods:

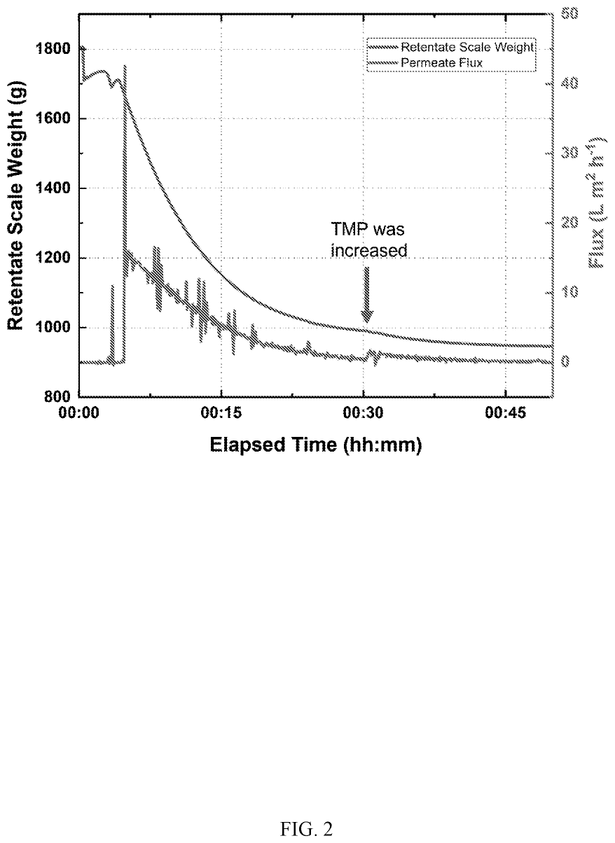

TFF Concentration (no draw solution): A concentrating TFF step was run without draw solution to concentrate a combined mixture of several dilute samples of ds oligonucleotide-containing solutions. The experimental parameters are listed in Table 1. The TMP was increased from 40 to 42 psi after about 30 min to prolong the permeate flux as long as possible, and the experiment was ended when the permeate flux was zero at the highest TMP value. The retentate scale weight and permeate flux data over time are shown in FIG. 2. The concentration of the final ds oligonucleotide retentate solution was measured to be 101 mg/mL. The oligonucleotide was a sodium salt duplex consisting of a 36-nucleotide sense strand containing GalNAc sugars on positions 28-30 (SEQ ID NO:3), complexed to a 22-nucleotide antisense strand (SEQ ID NO:4).

Density and Concentration Measurements: Concentrated samples were filtered with 0.22 μM filters (Millipore, Burlington, MA; Steriflip 50 mL with 0.22 μM Durapore PVDF membranes) prior to density and concentration measurements. Density measurements were recorded using a DMA 4100 M Density Meter (Anton Paar, Ashland, Virginia) at 20° C. Density measurements were used to make gravimetric dilutions of siRNA solutions for concentration measurements.

UV Assay for Oligonucleotide Concentration Measurements: A UV assay was used to determine the concentration of ds oligonucleotide-containing solutions (e.g., siRNA) at 258 nm using an extinction coefficient of 5.48×105 M-1 cm-1. Samples were diluted to about 0.02 mg/mL to be within the linear range of Beer-Lambert's law for measurement using a 1 cm path length cuvette. Concentrations reported were an average from triplicate dilutions of each sample and were corrected for duplex purity, with water as reference. The following equation was used to generate an average concentration value, where Abs was the average absorbance value at 258 nm from triplicate measurements, V was the volume of the of the first dilution, density was the measured density of the concentrated solution, MW was the molecular weight of the free acid form of the molecule (20675 g/mol), DF was the dilution factor of the second dilution, W was the sample weight of the concentrated sample, MEC was the molar extinction coefficient (548000 M⁻¹ cm⁻¹), Path Length was 1 cm, and Purity was determined from non-denaturing UHPLC (98.7%).

$$Assay ( mg / ml ) = \frac{Abs \times V \times Density \times M W \times DF}{W \times MEC \times Path Length} \times \frac{1000 mg / g}{1000 mL / L} \times Purity .$$

TABLE 1
TFF Parameters Used for TFF Without Draw Solution.
Parameter

| Membrane | Manufacturer | Pall (T-series) |
|---|---|---|
| | Material | Omega polyethersulfone |
| | | (PES) |
| | Molecular-weight cut-off | 3 |
| | (MWCO) (kDa) | |
| | Surface area per membrane | 0.1 |
| | (m²) | |
| | Total surface area used (m²) | 0.3 |
| | Pump flow rate (L/min) | 1 |
| | TMP (psi) | 40, 42 |

Results: With no draw solution, retentate scale weight and permeate flux decreased over time (FIG. 2). TMP was increased from 40 psi to 42 psi after 30 min. The ds oligonucleotide concentration of the final retentate was 101 mg/mL.

### Example 2: TFF of a Ds Oligonucleotide-Containing Solution with a Draw Solution

Purpose: To improve the final concentration of a ds oligonucleotide-containing solution over typical TFF.

### Methods:

TFF Concentration (with draw solution): The same general procedure was followed here as described above with variation in specific parameters noted for each example. The system and membranes were flushed and equilibrated with H₂O prior to use and then loaded with ds oligonucleotide-containing solution in the retentate reservoir. As with Example 1, the oligonucleotide was a sodium salt duplex consisting of a 36-nucleotide sense strand containing GalNAc sugars on positions 28-30 (SEQ ID NO:3), complexed to a 22-nucleotide antisense strand (SEQ ID NO:4).

In addition, a draw solution was added to the permeate collection reservoir. Tubing was connected from a draw solution to a permeate inlet valve on a membrane cassette holder, feeding through a peristaltic pump. The draw solution was flushed through the lines and permeate side of the membrane, and the permeate balance was tared. The concentrating experiment was run at a specified TMP and flow rate until either a minimum volume was reached in the retentate reservoir or until there was no measurable permeate flow for 30 min. The draw solution was circulated at a flow rate of 10 mL/min from the start of the experiment unless otherwise noted. The primary retentate was collected, followed by washes with H₂O. The system was sanitized and stored in a NaOH solution.

Here, three different concentrations of a NaCl draw solution were used to concentrate ds oligonucleotide-containing solutions via TFF, in comparison with just H₂O as a draw solution. The concentrations of the NaCl solutions were 0, 10, 100 and 500 mM (with ionic strengths of 0, 0.01, 0.1 and 0.5 mol/L, respectively). Besides the draw solution concentration, all other experimental parameters were held constant, according to Table 2, and the same general TFF protocol was followed as described above. For the 0, 10 and 100 mM samples, runs were stopped when the retentate scale weight remained constant for 30 min. For the 500 mM sample, runs were stopped when the minimum reservoir volume was reached or when the retentate scale weight remained constant for 30 min.

TABLE 2
TFF Parameters Used for TFF With Draw Solution.
Parameter

| Membrane | Manufacturer | Sartorius |
|---|---|---|
| | Material | Hydrosart ® |
| | | (regenerated cellulose) |
| | MWCO (kDa) | 2 |
| | Surface area per | 0.02 |
| | membrane (m²) | |
| | Total surface | 0.06 |
| | area used (m²) | |
| | Pump flow rate (L/min) | 0.5 |
| | TMP (psi) | 30 |
| Starting ds | Volume (mL) | 164 |
| Oligonucleotide- | Concentration (mg/mL) | 72 |
| Containing Solution | | |
| | Draw solution volume (mL) | 164 |

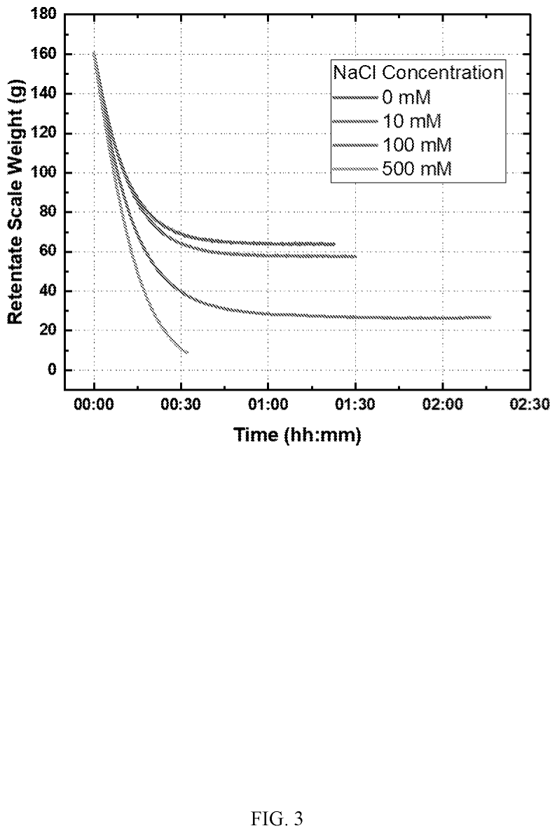

Results: The effect of varying the draw solution was highlighted by an increase in final ds oligonucleotide concentration with increasing draw solution concentration and ionic strength, as shown below in Table 3. The trend correlated with a faster decrease in the retentate scale weight taken of the bulk solution during the TFF process (FIG. 3), leading to an increase in H₂O removed from the ds oligonucleotide-containing solution. 100 mM-containing and 500 mM-containing draw solutions resulted in final ds oligonucleotide concentrations greater than those achieved in Example 1 without a draw solution.

TABLE 3
Effect of Concentration and Ionic Strength
of Various NaCl Draw Solutions.

| NaCl Concentration | NaCl Ionic Strength | Final ds Oligonucleotide |
|---|---|---|
| (mM) | (mol/L) | Concentration (mg/mL) |
| 0 | 0 | 85 |
| 10 | 0.01 | 91 |
| 100 | 0.1 | 122 |
| 500 | 0.5 | 153 |

### Example 3: TFF of a Ds Oligonucleotide-Containing Solution with an Alternative Draw Solution

Purpose: To assess the effects of varying draw solution concentration and ionic strength on the final concentration of a ds oligonucleotide-containing solution during TFF.

### Methods:

TFF Concentration (with a draw solution): A 2 M (2000 mM, with ionic strength of 2 mol/L) NaCl draw solution was used to concentrate a ds oligonucleotide-containing solution via TFF, using the parameters listed in Table 4. Like Examples 1 and 2, the ds oligonucleotide was a sodium salt duplex consisting of a 36-nucleotide sense strand containing GalNAc sugars on positions 28-30 (SEQ ID NO:3), complexed to a 22-nucleotide antisense strand (SEQ ID NO:4).

TABLE 4
TFF Parameters Used for TFF With Draw Solution.
Parameter

| Membrane | Manufacturer | Sartorius |
|---|---|---|
| | Material | Hydrosart ® |
| | | (regenerated cellulose) |
| | MWCO (kDa) | 2 |
| | Surface area per | 0.02 |
| | membrane (m²) | |
| | Total surface | 0.06 |
| | area used (m²) | |
| | Pump flow rate (L/min) | 0.43 |
| | TMP (psi) | 45 |
| Starting ds | Volume (mL) | 360 |
| Oligonucleotide- | Concentration (mg/mL) | 95 |
| Containing Solution | | |
| | Draw solution volume (mL) | 500 |

Results: The ds oligonucleotide concentration of the final retentate solution was measured to be 308 mg/mL. While the results in Example 2 show that the draw solution concentration and ionic strength lead to an increase in ds oligonucleotide final concentration, this example shows an even higher concentration was achieved by using a greater starting amount of material, a higher concentration and ionic strength of NaCl draw solution, and higher TMP.

### Example 4: TFF of a Ds Oligonucleotide-Containing Solution with an Alternative Draw Solution and Alternative Draw Solution Pump Start

Purpose: To assess changing conditions of TFF to determine the effect to concentrate a ds oligonucleotide-containing solution.

### Methods:

TFF Concentration (with a draw solution): A 1.8 M Arginine HCl draw solution was used to concentrate a ds oligonucleotide-containing solution (i.e., the oligonucleotide was a sodium salt duplex consisting of a 36-nucleotide sense strand containing GalNAc sugars on positions 28-30 (SEQ ID NO:3), complexed to a 22-nucleotide antisense strand (SEQ ID NO:4)) via TFF, using the parameters listed below in Table 5. Here, the TFF was run initially without the draw solution until the permeate flux was 0 and the retentate scale weight was no longer decreasing, then the draw solution pump was turned on.

TABLE 5
TFF Parameters used for TFF With Draw Solution.
Parameter

| Membrane | Manufacturer | Pall (T-series) |
|---|---|---|
| | Material | Omega |
| | | polyethersulfone |
| | | (PES) |
| | MWCO (kDa) | 3 |
| | Surface area per | 0.1 |
| | membrane (m²) | |
| | Total surface | 0.3 |
| | area used (m²) | |
| | Pump flow rate (L/min) | 0.5 |
| | TMP (psi) | 40, 42 |
| Starting ds | Volume (mL) | 500 |
| Oligonucleotide- | Concentration (mg/mL) | 85* |
| Containing Solution | | |
| | Draw solution volume (mL) | 500 |

*concentration measured from an older method using SoloVPE.

Results: The effect of the draw solution is apparent by the immediate steep decrease in the retentate scale weight after the draw solution was added. The ds oligonucleotide concentration of the final retentate solution was measured to be 228 mg/mL.

### Example 5: Effect of Varying Salts in Draw Solution for TFF

Purpose: To assess changing the type of salt in a draw solution to determine the effect to concentrate a ds oligonucleotide-containing solution.

### Methods:

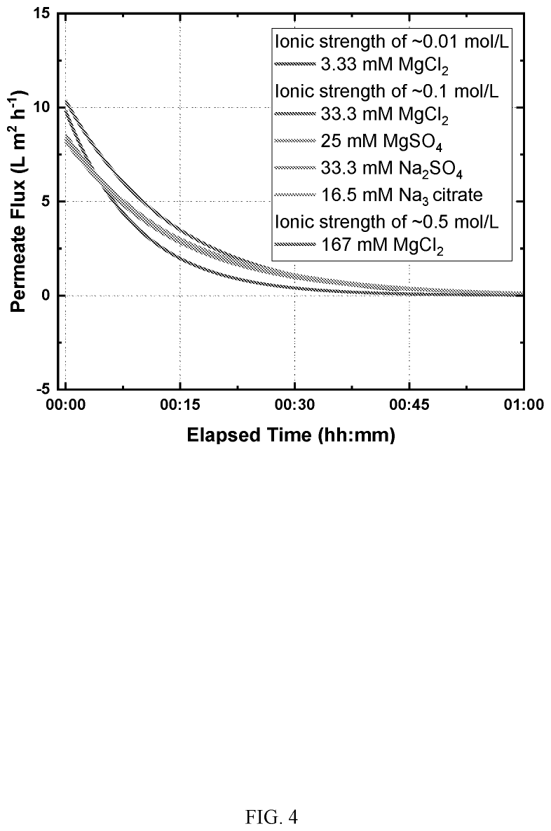

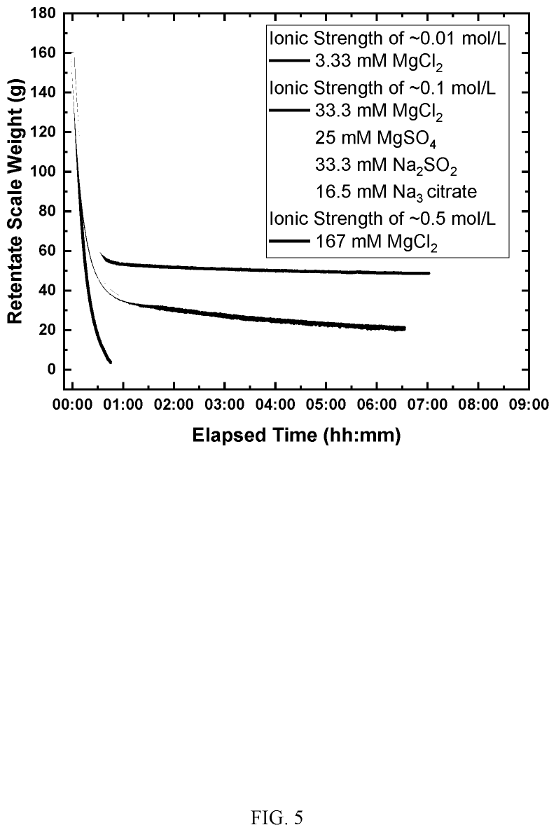

TFF Concentration: Various salts were used as draw solutions to concentrate a ds oligonucleotide-containing solution (i.e., the oligonucleotide was a sodium salt duplex consisting of a 36-nucleotide sense strand containing GalNAc sugars on positions 28-30 (SEQ ID NO:3), complexed to a 22-nucleotide antisense strand (SEQ ID NO:4)) via TFF, using the parameters listed in Table 6. The different draw solutions and concentrations are listed in Table 7. Besides the draw solution, all other experimental parameters were held constant for comparison. Experimental conditions were the same as those in Example 1, and the 0 mM control sample described in Example 1 was used as a comparison to determine if the various draw solutions could achieve a higher concentration. The final siRNA concentrations are listed in Table 6, the permeate flux data is plotted in FIG. 4, and the retentate scale weight data is shown in FIG. 5.

The runs were ended when the retentate scale weight remained constant for 30 min or when the minimum reservoir volume was reached (for the 167 mM MgCl₂ draw solution sample). Data was adjusted to start at the same weight at a time of 0 to account for slight variations in the delay time between the start of data collection and the start of the run for each TFF experiment.

TABLE 6
TFF Parameters used for TFF With Draw Solution.
Parameter

| Membrane | Manufacturer | Sartorius |
|---|---|---|
| | Material | Hydrosart ® |
| | | (regenerated cellulose) |
| | MWCO (kDa) | 2 |
| | Surface area per | 0.02 |
| | membrane (m²) | |
| | Total surface | 0.06 |
| | area used (m²) | |
| | Pump flow rate (L/min) | 0.5 |
| | TMP (psi) | 30 |
| Starting ds | Volume (mL) | 164 |
| Oligonucleotide- | Concentration (mg/mL) | |
| Containing Solution | | 72 |
| | Draw solution volume (mL) | 164 |

TABLE 7
Draw Solutions and Final ds Oligonucleotide Concentrations.

| | | | Final ds |
|---|---|---|---|
| | Draw Solution | Draw Solution | Oligonucleotide |
| | Concentration | Ionic Strength | Concentration |
| Draw solution | (mM) | (mol/L) | (mg/mL) |
| Control (H₂O) | 0 | 0 | 81 |
| MgCl₂ | 3.33 | 0.00999 | 92 |
| MgCl₂ | 33.3 | 0.0999 | 111 |
| MgCl₂ | 167 | 0.501 | 136 |
| MgSO₄ | 25 | 0.1 | 102 |
| Na₂SO₄ | 33.3 | 0.0999 | 116 |
| Na₃C₆H₅O₇ | 16.5 | 0.099 | 110 |

Results: The 33.3 mM MgCl₂, 25 mM MgSO₄, Na₂SO₄, and 16.5 mM Na₃C₆H₅O₇ draw solutions had about the same ionic strength, as listed in Table 7, and reached relatively similar final ds oligonucleotide concentration values (varying from 102 mg/mL to 116 mg/mL). All draw solutions enabled greater ds oligonucleotide concentration than a control solution of H₂O, with the highest ionic strength solution reaching the greatest ds oligonucleotide concentration. While different salts affected the overall run time, as evidenced by the retentate scale data in FIG. 5, solutions with similar ionic strength had almost identical permeate flux values during the initial hour (FIG. 4).

### Example 6: High Molecular Weight Molecule in Draw Solution for TFF

Purpose: To assess using a high molecular weight molecule in a draw solution to determine the effect to concentrate a ds oligonucleotide-containing solution.

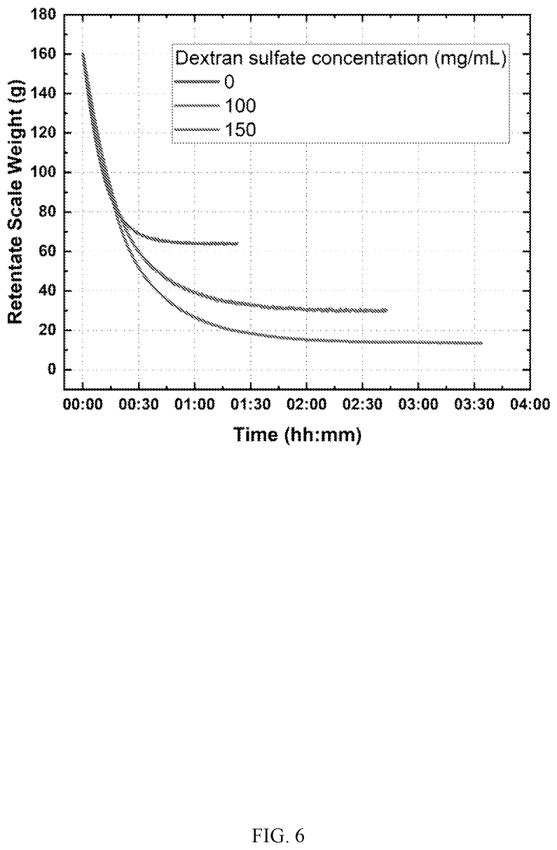

Methods: The sodium salt of dextran sulfate, a sulfated polysaccharide, was used as a draw solution to concentrate a ds oligonucleotide-containing solution (i.e., the oligonucleotide was a sodium salt duplex consisting of a 36-nucleotide sense strand containing GalNAc sugars on positions 28-30 (SEQ ID NO:3), complexed to a 22-nucleotide antisense strand (SEQ ID NO:4)) via TFF, using the parameters listed in Table 8. The average molecular weight of the dextran sulfate was ˜500 kDa, which should not be able to cross the TFF membrane with a MWCO of 2 kDa. Draw solutions with concentrations of 100 mg/mL and 150 mg/mL were tested. Besides the draw solution, all other experimental parameters were held constant for comparison. Experimental conditions were comparable to those in Example 1, and the 0 mM control sample described in Example 1 was used as a comparison to determine if the dextran sulfate draw solutions could achieve a higher ds oligonucleotide concentration. The final ds oligonucleotide concentrations are listed in Table 9, and the retentate scale weight data is shown in FIG. 6.

The runs were ended when the retentate scale weight remained constant for 30 min. Data was adjusted to start at the same weight at a time of 0 to account for slight variations in the delay time between the start of data collection and the start of the run for each TFF experiment.

TABLE 8
TFF Parameters used for TFF With Draw Solution.
Parameter

| Membrane | Manufacturer | Sartorius |
|---|---|---|
| | Material | Hydrosart ® |
| | | (regenerated cellulose) |
| | MWCO (kDa) | 2 |
| | Surface area per | 0.02 |
| | membrane (m²) | |
| | Total surface | 0.06 |
| | area used (m²) | |
| | Pump flow rate (L/min) | 0.5 |
| | TMP (psi) | 30 |
| Starting ds | Volume (mL) | 162 |
| Oligonucleotide- | Concentration (mg/mL) | 63* |
| Containing Solution | | |
| | Draw solution volume (mL) | 162 |

*Concentration measured from an older method using SoloVPE.

TABLE 9
Draw Solutions and Final ds Oligonucleotide Concentrations.

| | Final ds Oligonucleotide- |
|---|---|
| Dextran sulfate concentration | Containing Concentration |
| (mg/mL) | (mg/mL) |
| 0 | 81 |
| 100 | 130 |
| 150 | 153 |

Results: Both dextran sulfate-containing draw solutions enabled greater concentration than a control solution of H₂O.

### SEQUENCE LISTING

The following nucleotide and/or amino acid sequences are referred to in the disclosure above and are provided below for reference.

-Synthetic oligonucleotide 1 (36 nt)
SEQ ID NO: 1
UCAAAAUGGAAGGUUAUACAGCAGCCGAAAGGCUGC
-Synthetic oligonucleotide 2 (22 nt)
SEQ ID NO: 2
UGUAUAACCUUCCAUUUUGAGG
-Synthetic oligonucleotide 3 (36 nt)
SEQ ID NO: 3
[mUs][mC][mA][mA][mA][mA][mU][fG][fG][fA][fA][mG]
[mG][mU][mU][mA][mU][mA][mC][mA][mG][mC][mA][mG]
[mC][mC][mG][ademA-GalNAc][ademA-GalNAc][ademA-
GalNAc][mG][mG][mC][mU][mG][mC]
-Synthetic oligonucleotide 4 (22 nt)
SEQ ID NO: 4
[MePhosphonate-40-mUs][fGs][fUs][fA][fU][mA][fA]
[mC][mC][fU][mU][mC][mC][fA][mU][mU][mU][mU][mG]
[mAs][mGs][mG]

1. An apparatus for concentrating biomolecules in a solution, the apparatus comprising:
i. a first reservoir in fluid communication with a first side of a membrane, wherein the first reservoir is a retentate feed, and wherein the membrane has a molecular weight cutoff (MWCO) of <about 5 kDa;
ii. a second reservoir in fluid communication with a second opposing side of the membrane, wherein the second reservoir is a draw solution feed and a permeate collector;
iii. a first feed pump for cycling a retentate past the first side of the membrane;
iv. a second feed pump for cycling a draw solution or draw solution/permeate mixture past the second opposing side of the membrane;
V. a first balance with stir plate, wherein the first balance with stir plate is for stirring the retentate in the first reservoir and for measuring the retentate;
vi. a second balance with stir plate, wherein the second balance with stir plate is for stirring the draw solution or draw solution/permeate mixture in the second reservoir and for measuring the draw solution or draw solution/permeate mixture; and
vii. a transmembrane pressure (TMP) controller.

2. The apparatus of claim 1, wherein the MWCO is at least about 1 kDa.

3. The apparatus of claim 1, wherein the MWCO is at least about 2 kDa.

4. The apparatus of claim 1, wherein the membrane has a surface area from about 0.02 m² to about 0.1 m².

5. The apparatus of claim 1, wherein the membrane is a polyethersulfone (PES) membrane or a regenerated cellulose membrane.

6. A method of concentrating oligonucleotides in a solution, the method comprising the steps of:
a. cycling a first solution past a first side of a membrane, wherein the first solution is a retentate having a starting oligonucleotide concentration of <about 95 mg/mL and a pH between about 6 to about 7, and wherein the membrane has a molecular weight cutoff (MWCO) of <about 5 kDa;
b. cycling a second solution past a second opposing side of the membrane, wherein the second solution is a draw solution having a pH between about 6 to about 8 and an ionic strength greater than the first solution;
c. maintaining a transmembrane pressure (TMP) between about 30 psi to about 45 psi across the membrane; and
d. maintaining the cycling of the retentate and the permeate past the first and second opposing membrane sides, respectively, until the first solution has a final oligonucleotide concentration of at least about 100 mg/mL.

7. The method of claim 6, wherein the starting oligonucleotide concentration is at least about 20 mg/mL.

8. The method of claim 6, wherein the first solution is water.

9. The method of claim 6, wherein the MWCO is at least about 1 kDa.

10. The method of claim 6, wherein the MWCO is at least about 2 kDa.

11. The method of claim 6, wherein the membrane has a surface area from about 0.02 m² to about 0.1 m².

12. The method of claim 6, wherein the membrane is a polyethersulfone (PES) membrane or a regenerated cellulose membrane.

13. The method of claim 6, wherein the ionic strength of the second solution is between about 0.01 mol/L to about 2.0 mol/L.

14. The method of claim 6, wherein the second solution comprises a NaCl concentration between about 0.01 M to about 2.0 M.

15. The method of claim 6, wherein the second solution comprises a MgCl₂ concentration between 0.03 M about to about 2.0 M.

16. The method of claim 6, wherein the second solution comprises a dextran sulfate concentration between 100 mg/mL about to about 200 mg/mL.

17. The method of claim 6, wherein the oligonucleotide is single-stranded (ss).

18. The method of claim 6, wherein the oligonucleotide is double-stranded (ds).

19. The method of claim 6, wherein the first solution is flowed past the first side of the membrane at a rate from about 0.1 L/min to about 1.5 L/min.

20. The method of claim 6, wherein the second solution is flowed past the second opposing side of the membrane at a rate from about 1.0 L/min to about 15.0 L/min.

21. The method of claim 6, wherein the ionic strength of the second solution is maintained during the cycling.

22. The method of claim 6, wherein the ionic strength of the second solution is increased during the cycling.

23. The method of claim 22, wherein the ionic strength of the second solution is increased by about 1% to about 10% as compared to its initial ionic strength.

24. The method of claim 6, wherein the final oligonucleotide concentration is from about 150 mg/mL to about 200 mg/mL.

25. The method of claim 6, wherein the final oligonucleotide concentration is from about 200 mg/mL to about 250 mg/mL.

26. The method of claim 6, wherein the final oligonucleotide concentration is from about 250 mg/mL to about 300 mg/mL.

27. The method of claim 6, wherein the final oligonucleotide concentration is >300 mg/mL.
